(19) United States
(12) Patent Application Publication
(10) Pub. No.: US 2024/0123141 A1
(43) Pub. Date: Apr. 18, 2024

(54) SYSTEM AND METHOD FOR DETERMINING THE EFFECT OF INGESTION OF MEALS OF VARYING CARBOHYDRATE CONTENT

(71) Applicant: INSULET CORPORATION, Acton, MA (US)

(72) Inventors: Joon Bok LEE, Acton, MA (US); James CAUSEY, Simi Valley, CA (US); John D’ARCO, Wilmington, MA (US); Jason O'CONNOR, Acton, MA (US)

(21) Appl. No.: 18/482,414

(22) Filed: Oct. 6, 2023

Related U.S. Application Data
(60) Provisional application No. 63/379,089, filed on Oct. 11, 2022.

(51) Int. Cl. A61M 5/142; A61M 5/172; G16H 20/17; G16H 50/50

(52) U.S. Cl. CPC A61M 5/14244; A61M 5/1723; G16H 20/17; G16H 50/50; A61M 2005/1401; A61M 2005/14292; A61M 2005/14296

(57) ABSTRACT

Disclosed herein is a system and method for calculating an expected peak blood glucose level of user during a post-prandial period, based on a quantity of carbohydrates to be ingested. The user's expected peak blood glucose levels for slow, medium and fast acting carbohydrates are calculated. The results are presented to the user to allow the user to make an informed decision regarding the type of meal to be ingested.

### RELATED APPLICATIONS

This application claims the benefit of U.S. Provisional Patent Application No. 63/379,089, filed Oct. 11, 2022, the entire contents of which are incorporated herein by reference in its entirety.

### BACKGROUND

Insulin therapy carries several long-term risks. The risks include reduced insulin sensitivity, the need to increase insulin dosage amount and the insulin regimen complexity with time, an increase in severe hypoglycemia, and the potential increase in cardio-vascular events and even mortality. Therefore, it is desirable for people with diabetes to keep their insulin ingestion no higher than necessary.

A reduction in total insulin use carries significant benefits. For example, research has shown that the lowering of insulin levels can dramatically improve diabetes and the factors associated with metabolic syndrome including central obesity, high blood pressure, and elevated blood lipids, which are also risk factors associated with cardiovascular disease. Other conditions are also known to show improvement under the influence of reduced insulin levels

Therefore, diabetics are often advised by health care providers to alter their lifestyle to reduce the need to administer insulin. One method to reduce insulin use is by implementing behavioral changes, such as eating habits, to change to a diet lower in carbohydrates or comprising foods with lower glycemic indices.

Many people with diabetes receive all or a portion of their daily insulin needs via a wearable automatic drug delivery device, which may be part of an automated drug delivery (ADD) system that monitors the user's glucose levels and which controls the quantity and timing of insulin delivery to the user. The drug delivery device can be designed to deliver any type of liquid drug to a user. In specific embodiments, the drug delivery device can be, for example, an OmniPod® drug delivery device manufactured by Insulet Corporation of Acton, Massachusetts. The drug delivery device can be a drug delivery device such as those described in U.S. Pat. Nos. 7,303,549, 7,137,964, or U.S. Pat. No. 6,740,059, each of which is incorporated herein by reference in its entirety.

Such ADD systems typically have or regularly receive the information necessary to determine the effect of the ingestion of a particular meal on the user's blood glucose levels. Therefore, it would be useful for such ADD systems to be able to advise the user of the effect of specific proposed meals on the user's blood glucose levels to enable the user to make healthier food choices.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In one exemplary embodiment disclosed herein is a method to provide users with an estimate of the of the user's peak post-prandial glucose concentrations for different categories of meal or food glycemic indices, to promote healthier eating habits and to reduce the user's overall insulin needs.

In one aspect of the disclosed subject matter, the user's typical glucose excursion following the ingestion of carbohydrates is estimated based on a variety of factors. In addition, the bolus dose(s) of insulin required to address the expected glucose excursion may be determined. In a second aspect of the disclosed subject matter, the user may be presented with information regarding the estimated peaks of the glucose excursion based on ingestion of a quantity of carbohydrates having a low, medium, or high glycemic index. In addition, the user may be presented with the bolus(es) required to address the expected glucose excursion(s).

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 illustrates a functional block diagram of an exemplary system suitable for use with the devices disclosed herein.

FIG. 2 is a flowchart showing the process for estimating the user's expected peak post-prandial glucose levels based on the ingestion of slow-, medium- or fast-acting carbohydrates.

FIGS. 3(a-b) show exemplary user interface displays of an automated drug delivery system where the results of the disclosed method are presented to the user.

### DETAILED DESCRIPTION

Various embodiments of the present invention include systems and methods for delivering a medication to a user using a drug delivery device, either autonomously, or in accordance with a wireless signal received from an electronic device. In various embodiments, the electronic device may be a user device comprising a smartphone, a smart watch, a smart necklace, a module attached to the drug delivery device, or any other type or sort of electronic device that may be carried by the user or worn on the body of the user and that executes an algorithm that computes the times and dosages of delivery of the medication. Alternatively, the drug delivery device operates an algorithm stored on the drug delivery device itself, without reliance on a remote electronic device for delivering medicament to the user.

For example, the user device or drug delivery device may execute an “artificial-pancreas” (AP) algorithm that computes the times and dosages of delivery of insulin. The user device and/or drug delivery device may also be in communication with a sensor, such as a glucose sensor or a continuous glucose monitor (CGM), that collects data on a physical attribute or condition of the user, such as a glucose level. The sensor may be disposed in or on the body of the user and may be part of the drug delivery device or may be a separate device.

Alternatively, the drug delivery device may be in communication with the sensor in lieu of or in addition to the communication between the sensor and the user device. The communication may be direct (if, e.g., the sensor is integrated with or otherwise a part of the drug delivery device) or remote/wireless (if, e.g., the sensor is disposed in a different housing than the drug delivery device). In these embodiments, the drug delivery device contains computing hardware (e.g., a processor, memory, firmware, etc.) that executes some or all of the algorithm that computes the times and dosages of delivery of the medication.

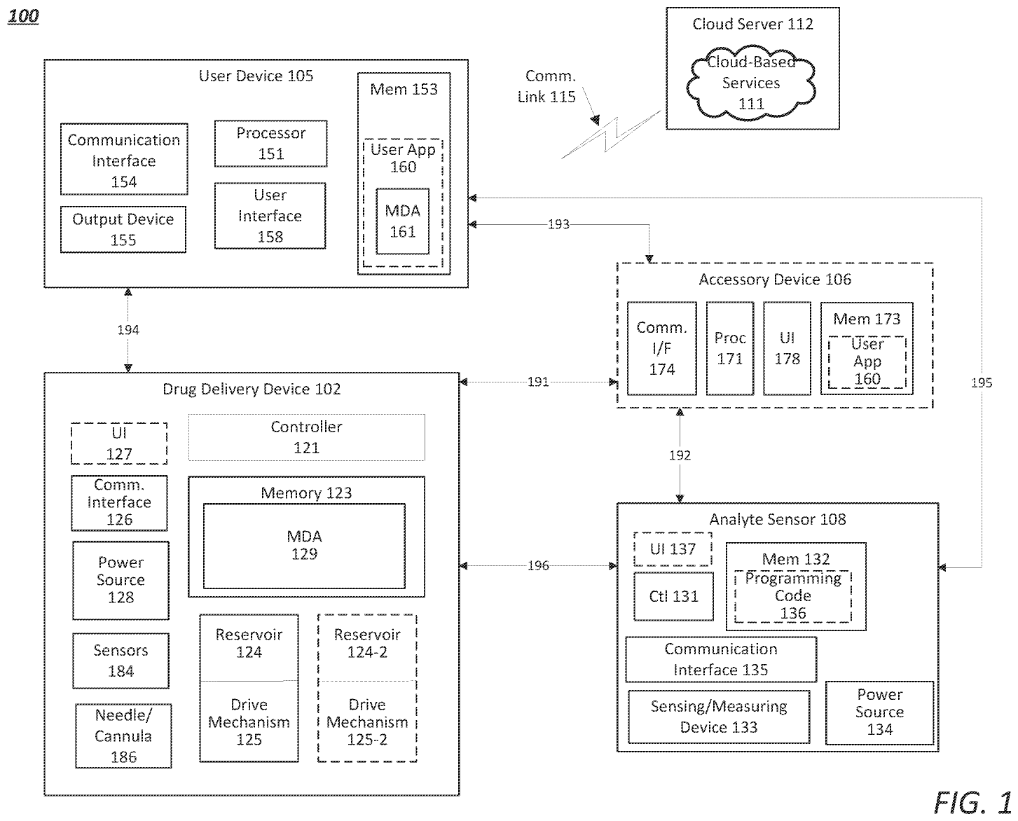

FIG. 1 illustrates a functional block diagram of an exemplary drug delivery system 100 suitable for implementing the systems and methods described herein. The drug delivery system 100 may implement (and/or provide functionality for) a medication delivery algorithm, such as an artificial pancreas (AP) application, to govern or control the automated delivery of a drug or medication, such as insulin, to a user (e.g., to maintain euglycemia—a normal level of glucose in the blood). The drug delivery system 100 may be an automated drug delivery system that may include a drug delivery device 102 (which may be wearable), an analyte sensor 108 (which may also be wearable), and a user device 105.

Drug delivery system 100, in an optional example, may also include an accessory device 106, such as a smartwatch, a personal assistant device, a smart insulin pen, or the like, which may communicate with the other components of system 100 via either a wired or wireless communication links 191-193.

### User Device

The user device 105 may be a computing device such as a smartphone, a smartwatch, a tablet, a personal diabetes management (PDM) device, a dedicated diabetes therapy management device, or the like. In an example, user device 105 may include a processor 151, device memory 153, a user interface 158, and a communication interface 154. The user device 105 may also contain analog and/or digital circuitry that may be implemented as a processor 151 for executing processes based on programming code stored in device memory 153, such as user application 160 incorporating medication delivery algorithm (MDA) 161 to manage a user's blood glucose levels and for controlling the delivery of the drug, medication, or therapeutic agent to the user, as well for providing other functions, such as calculating carbohydrate-compensation dosage, a correction bolus dosage and the like as discussed below. The user device 105 may be used to activate, deactivate, trigger a needle/canula insertion, program, adjust settings, and/or control operation of drug delivery device 102 and/or the analyte sensor 103 as well as the optional smart accessory device 106.

The processor 151 may also be configured to execute programming code stored in device memory 153, such as the user app 160. The user app 160 may be a computer application that is operable to deliver a drug based on information received from the analyte sensor 103, the cloud-based services 111 and/or the user device 105 or optional accessory device 106. The memory 153 may also store programming code to, for example, operate the user interface 158 (e.g., a touchscreen device, a camera or the like), the communication interface 154 and the like. The processor 151, when executing user app 160, may be configured to implement indications and notifications related to meal ingestion, blood glucose measurements, and the like. The user interface 158 may be under the control of the processor 151 and be configured to present a graphical user interface that enables the input of a meal announcement, adjust setting selections and the like as described herein.

In a specific example, when the user app 160 includes MDA 161, the processor 151 is also configured to execute a diabetes treatment plan (which may be stored in a memory) that is managed by user app 160. In addition to the functions mentioned above, when user app 160 is an AP application, it may further provide functionality to determine a carbohydrate-compensation dosage, a correction bolus dosage and determine a real-time basal dosage according to a diabetes treatment plan. In addition, as a MDA 161, user app 160 provides functionality to output signals to the drug delivery device 102 via communications interface 154 to deliver the determined bolus and/or basal dosages.

The communication interface 154 may include one or more transceivers that operate according to one or more radio-frequency protocols. In one embodiment, the transceivers may comprise a cellular transceiver and a Bluetooth® transceiver. The communication interface 154 may be configured to receive and transmit signals containing information usable by user app 160.

User device 105 may be further provided with one or more output devices 155 which may be, for example, a speaker or a vibration transducer, to provide various signals to the user.

### Drug Delivery Device

In various exemplary embodiments, drug delivery device 102 may include a reservoir 124 and drive mechanism 125, which are controllable by controller 121, executing a medication delivery algorithm (MDA) 129 stored in memory 123, which may perform some or all of the functions of the AP application described above, such that user device 105 may be unnecessary for drug delivery device 102 to carry out drug delivery and control. Alternatively, controller 121 may act to control reservoir 124 and drive mechanism 125 based on signals received from user app 160 executing on a user device 105 and communicated to drug delivery device 102 via communication link 194. Drive mechanism 125 may operate to longitudinally translate a plunger through the reservoir, so as to force the liquid drug through an outlet fluid port to needle/cannula 186. Alternatively, other types of drive mechanisms may be used.

In an alternate embodiment, drug delivery device 102 may also include an optional second reservoir 124-2 and second drive mechanism 125-2 which enables the independent delivery of two different liquid drugs. As an example, reservoir 124 may be filled with insulin, while reservoir 124-2 may be filled with glucagon, or Pramlintide, or GLP-1. In some embodiments, each of reservoirs 124, 124-2 may be configured with a separate drive mechanism 125, 125-2, respectively, which may be separately controllable by controller 121 under the direction of MDA 129. Both reservoirs 124, 124-2 may be connected to a common needle/cannula 186.

Drug delivery device 102 may be optionally configured with a user interface 127 providing a means for receiving input from the user and a means for outputting information to the user. User interface 127 may include, for example, light-emitting diodes, buttons on a housing of drug delivery device 102, a sound transducer, a micro-display, a microphone, an accelerometer for detecting motions of the device or user gestures (e.g., tapping on a housing of the device) or any other type of interface device that is configured to allow a user to enter information and/or allow drug delivery device 102 to output information for presentation to the user (e.g., alarm signals or the like).

Drug delivery device 102 includes a patient interface 186 for interfacing with the user to deliver the liquid drug. Patient interface may be, for example, a needle or cannula for delivering the drug into the body of the user (which may be done subcutaneously, intraperitoneally, or intravenously). Drug delivery device 102 may further include a mechanism for inserting the needle/cannula 186 into the body of the user, which may be integral with or attachable to drug delivery device 102. The insertion mechanism may comprise, in one embodiment, an actuator that inserts the needle/cannula 186 under the skin of the user and thereafter retracts the needle, leaving the cannula in place. The actuator may be triggered by user device 105 or may be a manual firing mechanism comprising springs or other energy storing mechanism, that causes the needle/cannula 186 to penetrate the skin of the user.

In one embodiment, drug delivery device 102 includes a communication interface 126, which may be a transceiver that operates according to one or more radio-frequency protocols, such as Bluetooth®, Wi-Fi, near-field communication, cellular, or the like. The controller 121 may, for example, communicate with user device 105 and an analyte sensor 108 via the communication interface 126.

In some embodiments, drug delivery device 102 may be provided with one or more sensors 184. The sensors 184 may include one or more of a pressure sensor, a power sensor, or the like that are communicatively coupled to the controller 121 and provide various signals. For example, a pressure sensor may be configured to provide an indication of the fluid pressure detected in a fluid pathway between the patient interface 186 and reservoir 124. The pressure sensor may be coupled to or integral with the actuator for inserting the patient interface 186 into the user. In an example, the controller 121 may be operable to determine a rate of drug infusion based on the indication of the fluid pressure. The rate of drug infusion may be compared to an infusion rate threshold, and the comparison result may be usable in determining an amount of insulin onboard (JOB) or a total daily insulin (TDI) amount. In one embodiment, analyte sensor 108 may be integral with drug delivery device 102.

Drug delivery device 102 further includes a power source 128, such as a battery, a piezoelectric device, an energy harvesting device, or the like, for supplying electrical power to controller 121, memory 123, drive mechanisms 125 and/or other components of drug delivery device 102.

Drug delivery device 102 may be configured to perform and execute processes required to deliver doses of the medication to the user without input from the user device 105 or the optional accessory device 106. As explained in more detail, MDA 129 may be operable, for example, to determine an amount of insulin to be delivered, JOB, insulin remaining, and the like and to cause controller 121 to activate drive mechanism 125 to deliver the medication from reservoir 124. MDA 129 may take as input data received from the analyte sensor 108 or from user app 160.

The reservoir 124 (and optionally reservoir 124-2) may be configured to store drugs, medications or therapeutic agents suitable for automated delivery, such as insulin, Pramlintide, GLP-1, co-formulations of insulin and GLP-1, glucagon, morphine, blood pressure medicines, arthritis drugs, chemotherapy drugs, fertility drugs, hormonal drugs, or the like.

Drug delivery device 102 may be a wearable device and may be attached to the body of a user, such as a patient or diabetic, at an attachment location and may deliver any therapeutic agent, including any drug or medicine, such as insulin or the like, to a user at or around the attachment location. A surface of drug delivery device 102 may include an adhesive to facilitate attachment to the skin of a user.

When configured to communicate with an external device, such as the user device 105 or the analyte sensor 108, drug delivery device 102 may receive signals over the wired or wireless link 194 from the user device 105 or from the analyte sensor 108. The controller 121 of drug delivery device 102 may receive and process the signals from the respective external devices as well as implementing delivery of a drug to the user according to a diabetes treatment plan or other drug delivery regimen.

### Accessory Device

Optional accessory device 106 may be, a wearable smart device, for example, a smart watch (e.g., an Apple Watch®), smart eyeglasses, smart jewelry, a global positioning system-enabled wearable, a wearable fitness device, smart clothing, or the like. Accessory device 106 may alternatively be a smart insulin pen that works with drug delivery device 102 in managing blood glucose and treating diabetes of a user. Similar to user device 105, the accessory device 106 may also be configured to perform various functions including controlling or communicating with drug delivery device 102. For example, the accessory device 106 may include a communication interface 174, a processor 171, a user interface 178 and a memory 173. The user interface 178 may be a graphical user interface presented on a touchscreen display of the smart accessory device 107. The memory 173 may store programming code to operate different functions of the smart accessory device 107 as well as an instance of the user app 160, or a pared-down version of user app 160 with reduced functionality. In some instances, accessory device 107 may also include sensors of various types.

### Analyte Sensor

The analyte sensor 108 may include a controller 131, a memory 132, a sensing/measuring device 133, an optional user interface 137, a power source/energy harvesting circuitry 134, and a communication interface 135. The analyte sensor 108 may be communicatively coupled to the processor 151 of the management device 105 or controller 121 of drug delivery device 102. The memory 132 may be configured to store information and programming code 136.

The analyte sensor 108 may be configured to detect one or multiple different analytes, such as glucose, lactate, ketones, uric acid, sodium, potassium, alcohol levels or the like, and output results of the detections, such as measurement values or the like. The analyte sensor 108 may, in an exemplary embodiment, be configured as a continuous glucose monitor (CGM) to measure a blood glucose values at a predetermined time interval, such as every 5 minutes, every 1 minute, or the like. The communication interface 135 of analyte sensor 108 may have circuitry that operates as a transceiver for communicating the measured blood glucose values to the user device 105 over a wireless link 195 or with drug delivery device 102 over the wireless communication link 108. While referred to herein as an analyte sensor 108, the sensing/measuring device 133 of the analyte sensor 108 may include one or more additional sensing elements, such as a glucose measurement element, a heart rate monitor, a pressure sensor, or the like. The controller 131 may include discrete, specialized logic and/or components, an application-specific integrated circuit, a microcontroller or processor that executes software instructions, firmware, programming instructions stored in memory (such as memory 132), or any combination thereof.

Similar to the controller 121 of drug delivery device 102, the controller 131 of the analyte sensor 108 may be operable to perform many functions. For example, the controller 131 may be configured by programming code 136 to manage the collection and analysis of data detected by the sensing and measuring device 133.

Although the analyte sensor 108 is depicted in FIG. 1 as separate from drug delivery device 102, in various embodiments, the analyte sensor 108 and drug delivery device 102 may be incorporated into the same unit. That is, in various examples, the analyte sensor 108 may be a part of and integral with drug delivery device 102 and contained within the same housing as drug delivery device 102 or an attachable housing thereto. In such an example configuration, the controller 121 may be able to implement the functions required for the proper delivery of the medication alone without any external inputs from user device 105, the cloud-based services 111, another sensor (not shown), the optional accessory device 106, or the like.

### Cloud-Based Services

Drug delivery system 100 may communicate with or receive services from a cloud server 122 providing cloud-based services 111. Services provided by cloud server 112 may include data storage that stores personal or anonymized data, such as blood glucose measurement values, historical IOB or TDI, prior carbohydrate-compensation dosage, and other forms of data. In addition, the cloud-based services 111 may process anonymized data from multiple users to provide generalized information related to TDI, insulin sensitivity, IOB and the like. The communication link 115 that couples the cloud server 112 to other components of system 100, for example, devices 102, 105, 106, 108 of system 100 may be a cellular link, a Wi-Fi link, a Bluetooth® link, or a combination thereof.

### Communication Links

The wireless communication links 115 and 191-196 may be any type of wireless link operating using known wireless communication standards or proprietary standards. As an example, the wireless communication links 191-196 may provide communication links based on Bluetooth®, Zigbee®, Wi-Fi, a near-field communication standard, a cellular standard, or any other wireless protocol via the respective communication interfaces 126, 135, 154 and 174.

### Operational Example

In an operational example, user application 160 implements a graphical user interface that is the primary interface with the user and may be used to activate drug delivery device 102, trigger a needle/cannula insertion, start and stop drug delivery device 102, program basal and bolus calculator settings for manual mode as well as program settings specific for automated mode (hybrid closed-loop or closed-loop).

User app 160, provides a graphical user interface 158 that allows for the use of large text, graphics, and on-screen instructions to prompt the user through the set-up processes and the use of system 100. It may also be used to program the user's custom basal insulin delivery profile, accept a recommended basal insulin delivery profile, check the status of drug delivery device 102, initiate bolus doses of insulin, make changes to a patient's insulin delivery profile, handle system alerts and alarms, or allow the user to switch between automated mode and manual mode.

User app 160 may be configured to operate in a manual mode in which user app 160 will deliver insulin at programmed basal rates and user-defined bolus amounts with the option to set temporary basal profiles. The controller 121 will also have the ability to function as a sensor-augmented pump in manual mode, using sensor glucose data provided by the analyte sensor 108 to populate the bolus calculator.

User app 160 may be configured to operate in an automated mode in which user app 160 supports the use of one or multiple target blood glucose values that may be adjusted manually or automatically by the system. For example, in one embodiment, target blood glucose values can range from 100-150 mg/dL, in 10 mg/dL increments, in 5 mg/dL increments, or other increments, but preferably 10 mg/dL increments. The experience for the user will reflect current setup flows whereby the healthcare provider may assist the user to program basal rates, glucose targets and bolus calculator settings. These in turn will inform the user app 160 for insulin dosing parameters. The insulin dosing parameters may be adapted over time based on the total daily insulin (TDI) delivered during each use of drug delivery device 102. A temporary hypoglycemia protection mode or an activity mode may be implemented by the user for various time durations in automated mode. With hypoglycemia protection mode, the algorithm reduces insulin delivery and is intended for use over temporary durations when insulin sensitivity is expected to be higher, such as during exercise or fasting.

The user app 160 (or MDA 129) may provide periodic insulin micro-boluses based upon past glucose measurements and/or a predicted glucose over a prediction horizon (e.g., 60 minutes). Glucose measurements and predicted glucose over a prediction horizon may be used to identify and compensate for missed manual meal boluses and mitigate prolonged hyperglycemia. The user app 160 uses a control-to-target strategy that attempts to achieve and maintain a set target glucose value or range, thereby reducing the duration of prolonged hyperglycemia and hypoglycemia.

In some embodiments, user device 105 and the analyte sensor 108 may not communicate directly with one another. Instead, data (e.g., blood glucose readings) from analyte sensor may be communicated to drug delivery device 102 via link 196 and then relayed to user device 105 via link 194. In some embodiments, to enable communication between analyte sensor 108 and user device 105, the serial number or other identifying information of the analyte sensor may be entered into user app 160 or scanned by user device 105.

User app 160 may provide the ability to calculate a suggested bolus dose through the use of a bolus calculator. The bolus calculator is provided as a convenience to the user to aid in determining the suggested bolus dose based on ingested carbohydrates, most-recent blood glucose readings (or a blood glucose reading if using fingerstick), programmable correction factor, insulin to carbohydrate ratio, target glucose value, and insulin on board (JOB). IOB is estimated by user app 160 taking into account any manual boluses and insulin delivered by the algorithm.

### DESCRIPTION OF EXEMPLARY EMBODIMENTS

Using the method disclosed herein, the glucose excursion for a particular user following the ingestion of carbohydrates can be estimated by a variety of factors, including, for example, the total quantity of carbohydrates in the ingested meal and the glycemic index (absorption rate) of the ingested meal. When a user requests a meal bolus from ADD system 100, an estimated maximum post-prandial peak of the user's blood glucose concentration can be determined based on the typical insulin action time of the delivered insulin over a post-prandial period, for example, 3 hours, and the varying meal absorption rates.

First, the typical impact of the user's glucose concentrations based on the current meal bolus, as well as the pre-existing insulin-on-board (IOB), can be calculated over the post-prandial period using Eqs. (1-2) as follows:

$$B ( k ) = \frac{CHO ( k )}{500 / TDI} - IOB ( k ) + \frac{G ( k ) - SP ( k )}{1800 / TDI} \qquad ( 1 )$$

where:
B(k) is the user's current required bolus to be administered at cycle (k). The required bolus can be calculated using a wide variety of methods. Eq. (1) is only provided as an exemplary method;
CHO(k) is the total carbohydrates that were ingested at cycle (k);
IOB(k) is the total insulin-on-board at cycle (k); and
“1800/TDI” is a correction factor for the user expressed as a ratio of mg/dL of change of the user's blood glucose level to units of insulin (TDI is total daily insulin). The “1800/TDI” is a tunable factor. “500/TD/” is an insulin to carbohydrate ratio expressed as a ratio of g of carbohydrates per units of delivered insulin (TDI is total daily insulin). The “500/TDI” is a tunable factor. The correction factor and insulin to carbohydrate ratio are tunable parameters that can be varied on a per-user basis;
G(k) is the glucose concentration for the current (k^{th}) cycle; and
SP(k) is the setpoint (glucose target) at the current (k^{th}) cycle set by the user or by ADD system 100 (or more particularly, MDA 129) and used for bolus calculations by MDA 129. Accordingly, when replacing the replacing the term “1800/TDI” with “a/TDI” and the term “500/TDI” by “b/TDI”, Equation (1) may be reformulated as:

$$B ( k ) = \frac{CHO ( k )}{b / TDI} - IOB ( k ) + \frac{G ( k ) - SP ( k )}{a / TDI} \qquad ( 1 a )$$

Eq. (2) provides an estimate of the user's blood glucose level decrease over an exemplary 36-cycle period (i.e., 3 hours assuming 5-minute cycles), based on the IOB, which includes both the insulin-on-board and the user's current requested (or system-determined) bolus:

$${G}_{IOB} ( k + 1 … 36 ) = 0.028 ( IOB ( k ) + B ( k ) ) \cdot \frac{1800}{TDI} \qquad ( 2 )$$

where:
“0.028” is a constant indicating the decrease in the effectiveness (or effective amount) of insulin every cycle, e.g. every 5-minute cycle. This is a tunable parameter that can be varied on a per-user basis and/or based on the cycle length; and
“36” is a constant indicating an exemplary 3-hour duration of insulin action, assuming 36 5-minute cycles in the 3-hour period. In various embodiments, the length of the post-prandial period (or hours of duration of insulin action) may be varied and/or the length of the cycles may be varied. Accordingly, when replacing in equation (2) the number of cycles per hour duration of insulin action by the term “c”, the constant “0.028” by the term “d”, Equation (2) may be reformulated as:

$${G}_{IOB} ( k + 1 … c ) = d \cdot ( IOB ( k ) + B ( k ) ) \cdot \frac{a}{TDI} \qquad ( 2 a )$$

The total insulin delivery in the k^{th} cycle, as a first estimate, can be estimated to be processed by the system over the next 36 cycles to 0, and the impact of this absorption can be modeled as a real time reduction in glucose concentration based on the user's estimated correction factor.

Next, the impact of the user's CHO ingestion on the user's glucose concentrations can also be calculated.

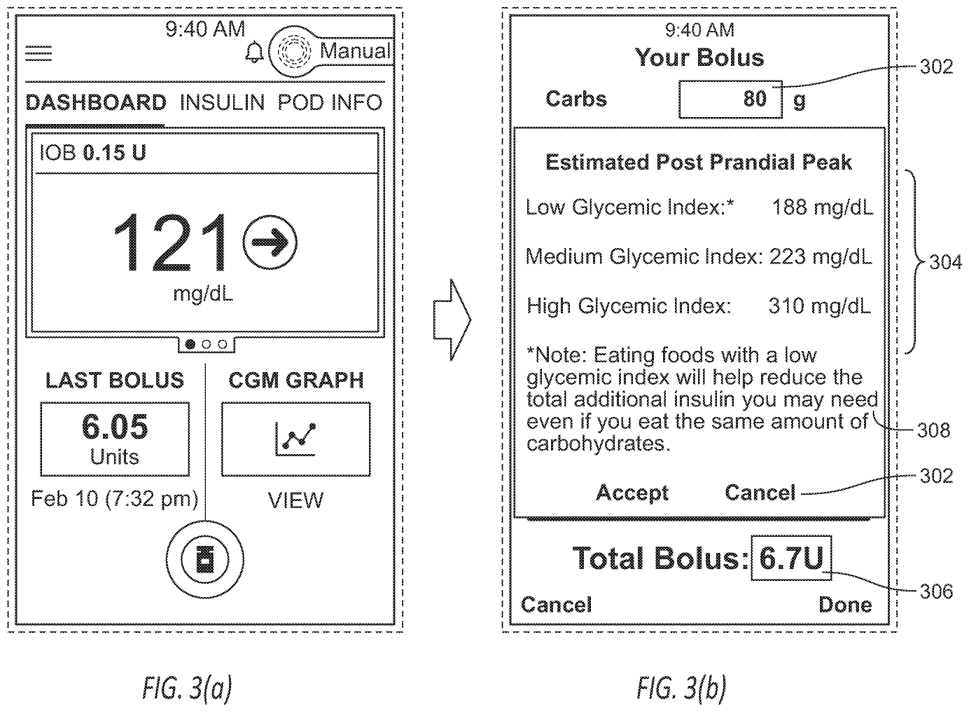

In one embodiment, this can be calculated in accordance with Eq. (3) and is based on an estimated linear absorption of each ingested meal based on fast (e.g., 2 hours), medium (e.g., 4 hours), and slow (e.g., 6 hours) absorbing carbohydrates, as follows:

$${G}_{CHO , N} ( k + 1 … N * 12 ) = \frac{1}{N * 12} ( {COB}_{N} ( k ) + CHO ( k ) ) \cdot 3.5 \qquad ( 3 )$$

where:
N is the number of hours selected for the absorption of fast (N=2), medium (N=4), and slow (N=6) acting carbohydrates. Nis a tunable parameter;
“3.5” is a tunable parameter indicating the approximate rise in mg/dL of the user's blood glucose level per 1 g of ingested carbohydrates;
CHO(k) is the proposed quantity of carbohydrates to be ingested for the current meal; and
COB_{N}(k) (i.e., “carbohydrates-on-board”) represents the impact of the user's residual carbohydrates;
and “12” is the number of cycles per hour. With reference to FIG. 3(b), the fast (e.g., 2 hours), medium (e.g., 4 hours), and slow (e.g., 6 hours) absorbing carbohydrates may correspond to the High, Medium, and Low Glycemic Index, respectively. In some embodiments, N may be between about 1 hour to about 3 hours for fast absorbing carbohydrates, more specifically between about 1.5 hours to about 2.5 hours. In some embodiments, N may be between about 3 hour to about 5 hours for medium absorbing carbohydrates, more specifically between about 1.5 hours to about 2.5 hours. In some embodiments, N may be between about 5 hour to about 7 hours for medium absorbing carbohydrates, more specifically between about 5.5 hours to about 6.5 hours. In other words, a food or meal with a high glycemic index contains fast-absorbing carbohydrates as it is closer to pure glucose on the glycemic index scale. When replacing in equation (3) the number of cycles per hour “12” by the term “e”, the constant “3.5” by the term “f”, Equation (3) may be reformulated as:

$${G}_{CHO , N} ( k + 1 … N * e ) = \frac{1}{N * e} ( {COB}_{N} ( k ) + CHO ( k ) ) \cdot f \qquad ( 3 a )$$

COB_{N}(k) may be calculated in accordance with Eq. (4), which demonstrates a vector multiplication of the series ingested carbohydrates, in grams, within the relevant time window (past N hours), by the series of values representing the linear decay curve over N hours, from 100% (1.0) to 0% (0.0):

$${COB}_{N} ( k ) = [ CHO ( k - N * 12 ) CHO ( k - N * 12 + 1 ) ⋯ ⋯ CHO ( k - 1 ) ] [ \begin{matrix}1 / ( N * 12 ) \\ 2 / ( N * 12 ) \\ 3 / ( N * 12 ) \\ ⋮ \\ ( N * 12 ) / ( N * 12 )\end{matrix} ] \qquad ( 4 )$$

where:
COB_{N}(k) is the carbohydrates on board based on an N hour decay curve, at the k^{th} cycle;
CHO(k) indicates the grams of carbohydrates that were ingested in the k^{th} cycle and N represents the duration, in hours, of the linear decay of the ingested carbohydrates. As explained above, the “12” is the number of cycles per hour “e”.

Finally, the user's peak glucose concentration during the post-prandial period (e.g., 3 hours) can be estimated, for each type of meal as follows:

G_{peak,N}(k)=G(k)+max(G_{CHO,N}(k+1 . . . N*12)−G_{IOB}(k+1 . . . N*12) (5)

where:
G_{peak,N}(k) is the user's peak glucose concentration over a post-prandial period (e.g., 3 hours; or another period could be used, such as 4 hours, or from cycle 1 to cycle 48, for “medium” absorbing type carbohydrates;
G(k) is the glucose concentration in the k^{th} cycle;
G_{CHO,N}(k+1 . . . N*12) is the impact on glucose concentration due carbohydrates that may be ingested in cycle 1 and takes N hours to be fully consumed over the course of an absorption time period (e.g., 2*12, or 2 hours (24 cycles) for fast absorbing carbohydrates), as can be calculated by Eq. (3); and
G_{IOB}(k+1 . . . N*12) is the impact on glucose concentration due to IOB over the course of N hours (e.g., 36 cycles or 12*3).

Note that Eqs. (3-5) may be calculated three times, once each for fast, medium and slow acting carbohydrates, by varying the value of N.

As would be realized by one of ordinary skill in the art, many variations in Eqs. (1-5) can be made and are contemplated to be within the scope of the disclosed embodiments; for example, the equations assume ADD system 100 has an MDA 129 which operates on 5-minute cycles. However, cycles of any duration could be used. In addition, the equations may assume that the peak blood glucose levels are predicted for a 3-hour period following ingestion of the meal; however, any time period could be used. For example, a post-prandial period of 4 hours could be used for “medium” absorbing carbohydrates. Lastly, all tunable parameters in the equations can be varied. In some embodiments, the cycles are between about 30 seconds to about 30 minutes, more specifically between about 1.5 minutes to about 10 minutes and in particular between about 3 minutes to about 9 minutes in duration. In some embodiments, the post-prandial period is between about 2 hours to about 4 hours in duration, more specifically between about 2.5 hours to about 3.5 hours in duration.

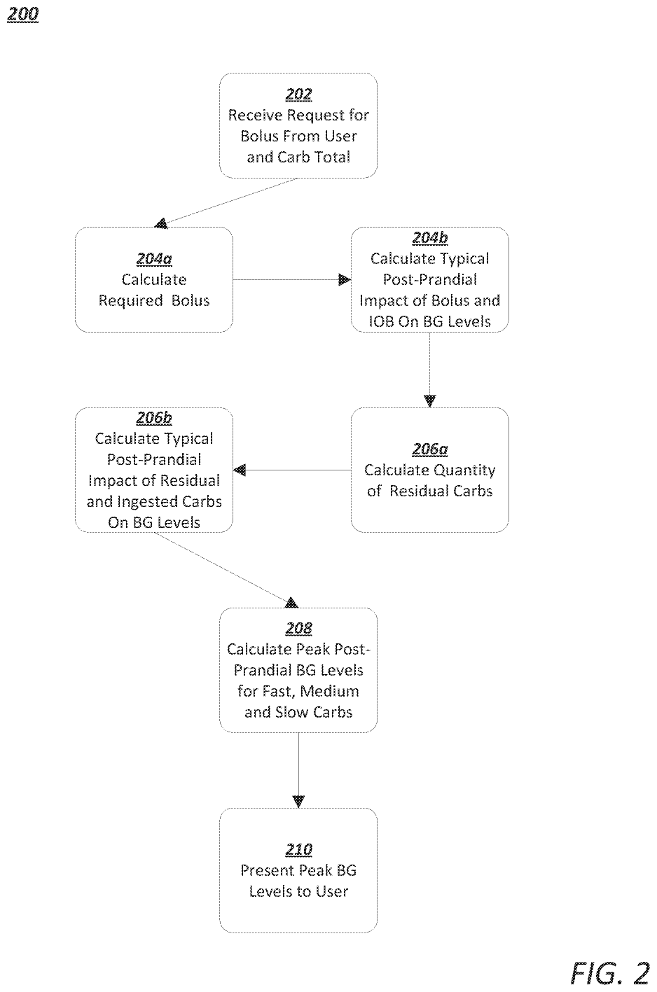

FIG. 2 is a flowchart showing process 200 for implementing the method discussed above. At step 202, a request is received from the user for a bolus calculation. The request includes the quantity of carbohydrates in the meal that the user intends to ingest. At step 204a, the user's required bolus is calculated (Eq. (1)) based on the number of carbohydrates the user entered and, at step 204b, a calculation (Eq. (2)) is made of the typical impact of the insulin-on-board and the bolus on the user's blood glucose levels during the post-prandial period (e.g., 3 hours). The “required bolus” may be the amount of liquid drug required to address excursions in the user's blood glucose levels based on the quantity of carbohydrates in the meal. Additionally or alternatively, the “required bolus” may be the amount of liquid drug required to return the user to their blood glucose target level during and/or at the end of the post-prandial period. At step 206a, the quantity of residual carbohydrates (i.e., carbs-on-board, representing carbohydrates remaining from previously ingested meals) present in the user's body during the post-prandial period is calculated (Eq. (4)). At step 206b, the typical effect of the residual carbohydrates and the proposed carbohydrates on the user's blood glucose levels during the post-prandial period is calculated (Eq. (3)). At step 208, the user's expected peak blood glucose level during the post-prandial period is calculated (Eq. (5)) based on the difference between the expected rise in the user's blood glucose levels based on the carbohydrates to be ingested, calculated at step 206b, and the reduction in the user's blood glucose level due to the insulin on board, calculated at step 204b. At step 210, the user's expected peak blood glucose levels, calculated at step 208, are presented to the user. Peak blood glucose levels for slow-, medium-, and fast-acting carbohydrates are presented. Accordingly, in some embodiments, multiple peak glucose levels may be presented to user, in particular at least two of the peak glucose levels for fast, medium and slow absorbing carbohydrates may be presented to the user, in particular all three of the peak glucose levels for fast, medium and slow absorbing carbohydrates may be presented to the user.

In the second aspect of the disclosed embodiments, the peak blood glucose levels of the user during the post-prandial period are presented to the user in a user interface 158 of user device 105 as generated by user app 160. The user may navigate from a default display of user app 160, shown in FIG. 3(a) to the display shown in FIG. 3(b). The display shown in FIG. 3(a) may be any user interface page of user app 160. The display shown in FIG. 3(b) includes field 302 wherein the user enters the quantity of carbs in an intended meal. In one exemplary embodiment, the user does not enter the estimated glycemic index for the food or meal the user is eating, hence the algorithm is unaware of whether the intended meal contains low, medium, or high glycemic index carbohydrates, and the expected peak blood glucose levels for all three types of meals are shown in area 304 of the display. In an alternative embodiment, the user may select an estimated category of glycemic index for the food or meal the user is eating (or about to eat). For example, the user may select “High Glycemic Index” on the screen shown in FIG. 3(b), or on a different or underlying screen of the bolus calculator. Accordingly, in some embodiments the display comprises a field for the glycemic index of the carbohydrates to be ingested during the meal By selecting “High Glycemic Index,” the algorithm now knows that the food or meal the user is eating (or about to eat) will be absorbed quickly, and may correspond to fast absorbing carbohydrates mentioned above, having an estimated digestion or post-prandial period of 2 hours (N=2). Accordingly, the algorithm may determine that the calculated bolus (e.g., 6.7 U in FIG. 3(b)) should be delivered immediately to compensate for the amount of fast-absorbing carbohydrates. Alternatively, if the user selects “Medium Glycemic Index” or “Low Glycemic Index,” the algorithm can adjust the bolus delivery timing and possibly the bolus delivery amount. For example, rather than delivering the entire bolus immediately (as may be done with High Glycemic Index foods or meals), the algorithm may deliver a portion of the bolus now (e.g., ½ or ⅔ of the bolus for a Medium Glycemic Index selection; or ⅓ for a Low Glycemic Index selection) and a portion or portions of the remaining bolus later (e.g., ½ or ⅓ of the remaining bolus 120 minutes later for a Medium Glycemic Index selection; or ⅔ of the remaining bolus 150 minutes later for a Low Glycemic Index selection; or ⅓ of the remaining bolus 120 minutes later and the remaining ⅓ 210 minutes after that for a Low Glycemic Index selection). Accordingly, in some embodiments, the bolus amount, bolus timing and/or bolus split are based on the selection of the Glycemic Index, in particular wherein a lower Glycemic Index leads to lower bolus amount, higher bolus split and/or later bolus timing. A “lower Glycemic Index” relates to slower absorbing carbohydrates. These portion amounts and timings are exemplary and other portion amounts and timings may be used. Before delivering any remaining portions of a bolus, the algorithm may determine whether the user's blood glucose has peaked already (as determined, for example, based on regular input from a glucose sensor, e.g., every 5 minutes), and if the user's blood glucose has already peaked, the remaining bolus may not be delivered. For example, if the user's blood glucose has stopped increasing and has started to drop, based on inputs from a glucose sensor, the algorithm may determine that a sufficient amount of insulin has already been delivered, and the remaining portion(s) of the bolus may not be delivered to the user.

With further reference to FIG. 3(b), the total bolus (in units of insulin) required to address the user's blood glucose excursion during the post-prandial period is shown in field 306 and is calculated in accordance with Eq. (1) or an algorithm in MDA 129. Instructional message 308 may be provided to the user informing the user that eating foods with a lower glycemic index will lower the additional insulin which may be needed to address the glucose excursion. The user may be presented with “Accept” and “Decline” buttons 310 to indicate to user app 160 whether the user intends to proceed with the proposed meal or not. Alternatively, as explained above, the user may select one of the glycemic indices to indicate to the algorithm what type of food or meal the user is eating (or about to eat). Additionally, or alternatively, the user may select one of the Low, Medium, or High Glycemic Index labels (or an information icon that may be next to such labels) and a user interface may be presented to the user showing what particular types of foods correspond to the category of glycemic indices. For example, the user may select the “High Glycemic Index” label, or an information icon next to such label, and a user interface may indicate to the user exemplary foods (via text and/or pictures) having a high glycemic index, such as pasta, rice, bread, a milkshake, or candy, for example.

As would be realized by one of skill in the art, the displays shown in FIGS. 3(a-b) are only exemplary in nature. Displays with other layouts or information presented on the displays are contemplated to be within the scope of the disclosed embodiments.

The described process 200 may be part of MDA 129 which may execute on drug delivery device 102, on user device 105, or partially on drug delivery device 102 and partially on user device 105. The information regarding the peak blood glucose levels may be presented to the user via user interface 158 on user device 105. The information may also be presented to the user via interface 178 on accessory device 106 which may be, for example, a smartwatch or other smart accessory device.

As would be realized by one of skill in the art, many variations on the embodiments disclosed herein are possible. In particular, varying the models and constants used in the models are contemplated to be the within the scope of the invention and the invention is not meant to be limited by the specific embodiments disclosed herein.

The following examples pertain to various embodiments disclosed herein for the needle insertion/reduction mechanism for use with an automatic drug delivery system.

Example 1 is a first embodiment of the invention directed to a method comprising predicting, using one or more models, the blood glucose level of user of an automatic drug livery system for a post-prandial period, identifying one or more predicted peak glucose readings during the post-prandial period and presenting, to the user, the predicted peak blood glucose readings. In some embodiments, the predicted peak blood glucose readings may be presented to the user in the form of a line graph and/or in the form of a text box.

Example 2 is an extension of Example 1, or any other example disclosed herein, the method further comprising accepting (i.e. receiving), as input from the user, a quantity of carbohydrates to be ingested during the meal and calculating a recommended bolus dose of a liquid drug required to address excursions in the user's blood glucose levels based on the quantity of carbohydrates in the meal.

Example 3 is an extension of Example 2, or any other example disclosed herein, wherein the one or more models includes an IOB model for determining the effect on the blood glucose levels of the user due to insulin-on-board for the post-prandial period.

Example 4 is an extension of Example 3, or any other example disclosed herein, wherein the insulin-on-board includes insulin in the body of the user from previous basal or bolus doses of insulin and insulin introduced via the recommended bolus dose.

Example 5 is an extension of Example 4, or any other example disclosed herein, wherein the one or more models includes a CHO model for determining the effect on the blood glucose levels of the user due to carbohydrates for the post-prandial period.

Example 6 is an extension of Example 5, or any other example disclosed herein, wherein the CHO model accounts for previously ingested carbohydrates and carbohydrates to be ingested during the meal.

Example 7 is an extension of example 6, or any other example disclosed herein, wherein the CHO model is evaluated for slow, medium and fast-acting carbohydrates.

Example 8 is an extension of Example 7, or any other example disclosed herein, wherein the predictions are made for each cycle of the medication delivery algorithm during the post-prandial period.

Example 9 is an extension of Example 8, or any other example disclosed herein, wherein the cycles of the medication delivery algorithm are 5 minutes in duration.

Example 10 is an extension of Example 8, or any other example disclosed herein, wherein the post-prandial period is 3 hours.

Example 11 is an extension of Example 8, or any other example disclosed herein, wherein identifying one or more predicted peak glucose readings during the post-prandial period comprises determining a predicted peak glucose reading based on each of slow-, medium- and fast-acting carbohydrates ingested during the meal.

Example 12 is an extension of Example 11, or any other example disclosed herein, wherein the peak glucose readings comprise the blood glucose level of the user at the time of the prediction plus a maximum of a difference, during each cycle of the medication delivery algorithm during the post-prandial period, between the rise in the user's blood glucose levels as predicted by the CHO model and a drop in the user's blood glucose levels as predicted by the IOB model.

Example 13 is an extension of claim 12, or any other example disclosed herein, wherein presenting the predicted blood glucose readings to the user comprises displaying, on user interface of a user device portion of the automatic drug delivery system, the predicted peak blood glucose level for slow-, medium- and fast-acting carbohydrates ingested during the meal.

Example 14 is an extension of Example 13, or any other example disclosed herein, wherein the display comprises a field wherein the user enters the quantity of carbohydrates to be ingested during the meal.

Example 15 is an extension of Example 13, or any other example disclosed herein, wherein the display informs the user of the required bolus.

Example 16 is a second embodiment of the invention is directed to an automatic drug delivery system comprising a processor and software, implementing a user application including a medication delivery algorithm executing on the processor, the software performing the functions of predicting, using one or more models, blood glucose levels of user of the automatic drug delivery system for a post-prandial period, identifying one or more predicted peak glucose readings during the post-prandial period and presenting, to the user, the predicted peak blood glucose readings.

Example 17 is an extension of Example 16, or any other example disclosed herein, the software performing the further functions of accepting, as input from the user, a quantity of carbohydrates to be ingested during the meal and calculating a recommended bolus dose of a liquid drug required to address excursions in the user's blood glucose levels based on the quantity of carbohydrates in the meal.

Example 18 is an extension of Example 17, or any other example disclosed herein, wherein the one or more models includes an IOB model for predicting the effect on the blood glucose levels of the user due to insulin-on-board for the post-prandial period and a CHO model for predicting the effect on the blood glucose levels of the user due to carbohydrates for the post-prandial period.

Example 19 is an extension of Example 18, or any other example disclosed herein, wherein the peak glucose readings comprise a blood glucose level of the user at the time of the prediction plus a maximum of a difference, during each cycle of the medication delivery algorithm during the post-prandial period, between a rise in the user's blood glucose levels as predicted by the CHO model and a drop in the user's blood glucose levels as predicted by the IOB model.

Example 20 is extension of Example 19, or any other example disclosed herein, wherein the peak blood glucose readings during the post-prandial period are predicted based on ingestion of meals comprising slow-, medium- and fast-acting carbohydrates ingested during the meal.

Example 21 is an extension of Example 20, or any other example is closed herein, further comprising a user device portion of the automatic drug delivery system including a display, wherein the software performs a further functions of providing a field in the display for the user to input the quantity of carbohydrates to be ingested during the meal, displaying the predicted blood glucose readings to the user on the display and displaying a recommended bolus based at least in part on the quantity of carbohydrates input by the user. Software related implementations of the techniques described herein may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific Ics (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

The present disclosure furthermore relates to computer programs comprising instructions (also referred to as computer programming instructions) to perform the aforementioned functionalities. The instructions may be executed by a processor. The instructions may also be performed by a plurality of processors for example in a distributed computer system. The computer programs of the present disclosure may be for example preinstalled on, or downloaded to the medicament delivery device, management device, fluid delivery device, e.g. their storage. The computer program may calculate the first portion and second portion to be delivered. The methods disclosed herein may be computer-implemented methods.

To those skilled in the art to which the invention relates, many modifications and adaptations of the invention may be realized. Implementations provided herein, including sizes, shapes, ratings, compositions and specifications of various components or arrangements of components, and descriptions of specific manufacturing processes, should be considered exemplary only and are not meant to limit the invention in any way. As one of skill in the art would realize, many variations on implementations discussed herein which fall within the scope of the invention are possible. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. The scope of the invention is defined by the claims which follow.

1. A method comprising:
predicting, using one or more models, blood glucose levels of a user of an automated drug delivery system for a predetermined post-prandial period after ingestion of a meal;
identifying one or more predicted peak blood glucose readings during the post-prandial period; and
presenting, to the user, the predicted peak blood glucose readings.

2. The method of claim 1 further comprising:
accepting, as input from the user, a quantity of carbohydrates of a meal; and
calculating a recommended bolus dose of a liquid drug required to address excursions in the user's blood glucose levels based on the quantity of carbohydrates in the meal.

3. The method of claim 2 wherein the one or more models includes an IOB model for predicting the effect on the blood glucose levels of the user due to insulin-on-board during the post-prandial period.

4. The method of claim 3 wherein the insulin-on-board includes insulin in the body of the user from previous basal or bolus doses of insulin.

5. The method of claim 4 wherein the one or more models includes a CHO model for predicting the effect on the blood glucose levels of user due to carbohydrates during the post-prandial period.

6. The method of claim 5 wherein the CHO model accounts for previously ingested carbohydrates and carbohydrates to be ingested during the meal.

7. The method of claim 5 wherein the CHO model is evaluated for slow, medium and fast-acting carbohydrates.

8. The method of claim 7 wherein the predictions are made for each cycle of a medication delivery algorithm during the post-prandial period.

9. The method of claim 8 wherein the cycles of the medication delivery algorithm are 5 minutes in duration.

10. The method of claim 8 wherein the post-prandial period is 3 hours.

11. The method of claim 8 wherein identifying one or more predicted peak glucose readings during the post-prandial period comprises determining a predicted peak glucose reading based on each of slow-, medium- and fast-acting carbohydrates ingested during the meal.

12. The method of claim 11 wherein the peak glucose readings comprise the blood glucose level of the user at the time of the prediction plus a maximum of a difference, during each cycle of the medication delivery algorithm during the post-prandial period, between a rise in the user's blood glucose levels as predicted by the CHO model and a drop in the user's blood glucose levels as predicted by the IOB model.

13. The method of claim 12 wherein presenting the predicted peak blood glucose readings to the user comprises displaying, on a user interface of a user device portion of the automatic drug delivery system, the predicted peak blood glucose levels for slow, medium and fast-acting carbohydrates ingested during the meal.

14. The method of claim 13 wherein the display comprises a field wherein the user enters the quantity of carbohydrates to be ingested during the meal.

15. The method of claim 13 wherein the display informs the user of the required bolus.

16. An automatic drug delivery system comprising:
a processor; and
software, implementing a user application including a medication delivery algorithm executing on the processor, the software performing the functions of:
predicting, using one or more models, blood glucose levels of a user of the automatic drug delivery system for a post-prandial period;
identifying one or more predicted peak blood glucose readings during the post-prandial period; and
presenting, to the user, the predicted peak blood glucose readings.

17. The system of claim 16, the software performing the further functions of:
accepting, as input from the user, a quantity of carbohydrates to be ingested during the meal; and
calculating a recommended bolus dose of a liquid drug required to address excursions in the user's blood glucose levels based on the quantity of carbohydrates in the meal.

18. The system of claim 17 wherein the one or more models includes an IOB model for predicting the effect on the blood glucose levels of the user due to insulin-on-board for the post-prandial period and a CHO model for predicting the effect on the blood glucose levels of user due to carbohydrates for the post-prandial period.

19. The system of claim 18 wherein the peak glucose readings comprise a blood glucose level of the user at the time of the prediction plus a maximum of a difference, during each cycle of the medication delivery algorithm during the post-prandial period, between a rise in the user's blood glucose levels as predicted by the CHO model and a drop in the user's blood glucose levels as predicted by the IOB model.

20. The system of claim 19 wherein the peak glucose readings during the post-prandial period are predicted based on ingestion of a meals comprising slow, medium and fast-acting carbohydrates ingested during the meal.

21. The system of claim 20, further comprising:
a user device portion of the automatic drug delivery system including a display;
wherein the software performs the further functions of:
providing a field on the display for the user to input the quantity of carbohydrates to be ingested during the meal;
displaying the predicted peak blood glucose readings to the user on the display; and
displaying a recommended bolus based at least in part on the quantity of carbohydrates input by the user.
